(19) United States
(12) Patent Application Publication
(10) Pub. No.: US 2002/0090622 A1
(43) Pub. Date: Jul. 11, 2002

(54) Chemical compounds

(76) Inventors: Monisola Adeokun, Macclesfield (GB); Helen Jean Ambrose, Macclesfield (GB); Carl John Cresswell, Macclesfield (GB); Adam Jeston Dudley, Wilmington (DE)

Correspondence Address: PILLSBURY WINTHROP LLP, 1600 TYSONS BOULEVARD, MCLEAN, VA, 22102, US

(21) Appl. No.: 09/925,731

(22) Filed: Aug. 10, 2001

Related U.S. Application Data
(60) Provisional application No. 60/226,909, filed on Aug. 23, 2000.

(51) Int. Cl. C12Q 1/68; C07H 21/04

(52) U.S. Cl. 435/6; 536/24.3

(57) ABSTRACT

This invention relates to polymorphisms in the human OATPC gene and corresponding novel allelic polypeptides encoded thereby. The invention also relates to methods and materials for analyzing allelic variation in the OATPC gene, and to the use of OATPC polymorphism in treatment of diseases with OATPC transportable drugs such as statins.

This invention relates to polymorphisms in the human OATPC gene and corresponding novel allelic polypeptides encoded thereby. The invention also relates to methods and materials for analysing allelic variation in the OATPC gene, and to the use of OATPC polymorphism in treatment of diseases with OATPC transportable drugs.

Na+-independent organic anion transporting polypeptide (OATP) C gene is a member of the OATP supergene family involved in multifunctional transport of organic anion. OATPC tranports the organic anion taurocholate, conjugated steroids: DHEAS, estradiol 17β-D-glucoronide and estrone-3-sulfate, eicosanoids: PGE₂, thromboxane B₂, leukotriene C₄, and E₄, and thyroid hormones T4 and T3^{1, 2}. OATPC has also been shown to be involved in the transport of xenobiotics, and drugs involved in lipid lowering e.g. statins¹. Statins have been refered to as a first-line therapy for patients with atherosclerotic vascular diseases. The OATPC gene and its product is also thought to be of importance in other diseases due to its transport of DHEAS an adrenal steroid which has been suggested to have positive neuropsychiatric, immune, and metabolic effects³. Due to the substrate specificity, location in the liver, and being exclusively expressed in the liver, Abe et al suggested that OATPC could be the predominant clearance mechanism of several endogenous and exogenous substrates in the liver. OATPC is the first human molecule reported to transport thyroid hormones².

This liver specific transporter may be useful in liver-specific drug delivery systems and liver-specific chemotherapy, bile acid formation and the pathogenesis of diseases such as cholestasis, hyperbilirubinemia and thyroid hormone resistance.

The OATPC gene (sometimes called OAPT2 in the literature) has been cloned by four different groups, annotated and published as EMBL accession numbers AB026257 (OATPC, 2452bp), AF205071(OATP2, 2830, ref 1), AJ132573(OATP2, 2778)⁴, and AF060500 (LST-1)². Polymorphism has been reported by Tamai⁵ which is Asn130Asp and Val174Ala although any functional effect was stated therein to be not clear. Konig (2000) J Biol Chem 275, 23161-23168 describes the genomic organisation of OATP 1, 2 and 8. International patent application WO 00/08157 describes human anion transporter genes and some polymorphisms.

All positions herein of polymorphisms in the OATPC polynucleotide relate to the position in one of SEQ ID NO 1 or 3-12 unless stated otherwise or apparent from the context.

All positions herein of polymorphisms in the OATPC polypeptide relate to the position in SEQ ID NO 2 unless stated otherwise or apparent from the context.

One approach is to use knowledge of polymorphisms to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The present invention is based on the discovery of polymorphisms in OATPC.

According to one aspect of the present invention there is provided a method for the detection of a polymorphism in OATPC in a human, which method comprises determining the sequence of the human at at least one polymorphic position and determining the status of the human by reference to polymorphism in the OATPC gene. Preferred polymorphic positions are one or more of the following positions:

A method for the detection of a polymorphism in OATPC in a human, which method comprises determining the sequence of the human at at least one of the following polymorphic positions:

positions 510, 696, 1299, 1312, 1347, 1561, 2028, 2327 and 2342 in sequence of the OATPC gene as defined by the position in SEQ ID NO: 1;

positions 400, 405, 488 and 643 in OATPC polypeptide defined by position in SEQ ID NO: 2;

positions 321 and 1332 defined by position in SEQ ID NO 3;

position 41 defined by position in SEQ ID NO 4;

positions 109 and 244 defined by position in SEQ ID NO 5;

positions 117 and 283 defined by position in SEQ ID NO 6;

positions 209 and 211 defined by position in SEQ ID NO 7;

positions 63 to 68 defined by position in SEQ ID NO 8;

position 53 defined by position in SEQ ID NO 9;

position 75 defined by position in SEQ ID NO 10;

position 162 defined by position in SEQ ID NO 11; and

positions 84 defined by position in SEQ ID NO 12.

The term human includes both a human having or suspected of having a OATPC mediated disease and an asymptomatic human who may be tested for predisposition or susceptibility to such disease. At each position the human may be homozygous for an allele or the human may be a heterozygote.

The term polymorphism includes single nucleotide substitution, nucleotide insertion and nucleotide deletion which in the case of insertion and deletion includes insertion or deletion of one or more nucleotides at a position of a gene.

The method for diagnosis is preferably one in which the sequence is determined by a method selected from amplification refractory mutation system and restriction fragment length polymorphism.

The status of the individual may be determined by reference to allelic variation at any one, two, three, four, five, six, seven, eight, nine or more positions.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| HMG-CoA | 3-hydroxy-3-methylglutaryl-coenzyme A |
| LCR | Ligase chain reaction |
| MASDA | Multiple allele specific diagnostic assay |
| NASBA | Nucleic acid sequence based amplification |
| OATP | Na+-independent organic anion transporting polypeptide |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

Table 1—Mutation Detection Techniques

General: DNA sequencing, Sequencing by hybridisation

Scanning: PTT*, SSCP, DGGE, TGGE, Cleavase, Heteroduplex analysis, CMC, Enzymatic mismatch cleavage

Hybridisation Based

Solid phase hybridisation: Dot blots, MASDA, Reverse dot blots,

Oligonucleotide arrays (DNA Chips)

Solution phase hybridisation: Taqman™—U.S. Pat. Nos. 5,210,015 & 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B1 (Zeneca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay * Note: not useful for detection of promoter polymorphisms.

Table 2—Signal Generation or Detection Systems

Fluorescence: FRET, Fluorescence quenching, Fluorescence polarisation—United Kingdom Patent No. 2228998 (Zeneca Limited)

Other: Chemiluminescence, Electrochemiluminescence, Raman, Radioactivity, Colorimetric, Hybridisation protection assay, Mass spectrometry

Table 3—Further Amplification Methods

SSR, NASBA, LCR, SDA, b-DNA

Preferred mutation detection techniques include ARMS™, ALEX™, COPS, Taqman, Molecular Beacons, RFLP, and restriction site based PCR and FRET techniques.

Particularly preferred methods include ARMS™ and RFLP based methods. ARMS™ is an especially preferred method.

In a further aspect, the diagnostic methods of the invention are used to assess the pharmacogenetics of a drug transportable by OATPC.

Assays, for example reporter-based assays, may be devised to detect whether one or more of the above polymorphisms affect transcription levels and/or message stability.

Individuals who carry particular allelic variants of the OATPC gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition and/or susceptibility of an individual to diseases mediated by OATPC. This may be particularly relevant in the development of hyperlipoproteinemia and cardiovascular disease and the present invention may be used to recognise individuals who are particularly at risk from developing these conditions.

In a further aspect, the diagnostic methods of the invention are used in the development of new drug therapies which selectively target one or more allelic variants of the OATPC gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the present invention there is provided a human OATPC gene or its complementary strand comprising a variant allelic polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 bases of the human OATPC gene and comprising an allelic variant selected from any one of the following:

| | | Position in | |
|---|---|---|---|
| Region | variant | SEQ ID NO | SEQ ID NO |
| Exon 4 | A | 510 | 1 |
| Exon 5 | T | 670 | 1 |
| Exon 5 | T | 696 | 1 |
| Exon 9 | G | 1299 | 1 |
| Exon 9 | A | 1312 | 1 |
| Exon 9 | A | 1347 | 1 |
| Exon 10 | C | 1561 | 1 |
| Exon 14 | C | 2028 | 1 |
| 3′UTR | Insert T | 2327 | 1 |
| 3′UTR | C | 2342 | 1 |
| Promoter | G | 321 | 3 |
| Promoter | C | 1332 | 3 |
| Intron 1 | A | 41 | 4 |
| Intron 2 | G | 109 | 5 |
| Intron 2 | G | 244 | 5 |
| Intron 3 | A | 117 | 6 |
| Intron 3 | A | 283 | 6 |
| Intron 4 | A | 209 | 7 |
| Intron 4 | A | 211 | 7 |
| Intron 4 | Deletion | 63 | 8 |
| | CTTGTA | | |
| Intron 6 | T | 53 | 9 |
| Intron 9 | Insert TTC | 75 | 10 |
| Intron 11 | Insert T | 162 | 11 |
| Intron 12 | C | 84 | 12 |

According to another aspect of the present invention there is provided a human OATPC gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting a OATPC gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a OATPC gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a OATPC gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers in linkage studies. This particularly applies to the polymorphisms of relatively high frequency. The OATPC gene is on chromosome 12p (as shown from a database search with the cDNA as a query sequence). Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a drug transportable by OATPC in which the method comprises:

i) detection of a polymorphism in OATPC in the human, which detection comprises determining the sequence of the human at one or more of the following positions:

positions 487, 510, 554, 670, 696, 819, 820, 1299, 1312, 1347, 1561, 2028, 2327 and 2342 in sequence of the OATPC gene as defined by the position in SEQ ID NO: 1;

positions 130, 152, 174, 241, 400, 405, 488 and 643 in OATPC polypeptide defined by position in SEQ ID NO: 2;

positions 321 and 1332 defined by position in SEQ ID NO 3;

position 41 defined by position in SEQ ID NO 4;

positions 109 and 244 defined by position in SEQ ID NO 5;

positions 117 and 283 defined by position in SEQ ID NO 6;

positions 209 and 211 defined by position in SEQ ID NO 7;

positions 63 to 68 defined by position in SEQ ID NO 8;

position 53 defined by position in SEQ ID NO 9;

position 75 defined by position in SEQ ID NO 10;

position 162 defined by position in SEQ ID NO 11; and

positions 84 defined by position in SEQ ID NO 12.

and determining the status of the human by reference to polymorphism in the OATPC gene; and

ii) administering an effective amount of the drug.

Preferably determination of the status of the human is clinically useful. Examples of clinical usefulness include deciding which statin drug or drugs to administer and/or in deciding on the effective amount of the statin drug or drugs. Statins already approved for use in humans include atorvastatin, cerivastatin, fluvastatin, pravastatin and simvastatin. The reader is referred to the following references for further information: Drugs and Therapy Perspectives (May 12, 1997), 9: 1-6; Chong (1997) Pharmacotherapy 17: 1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368. A preferred statin drug is compound 3a (S-4522) in Watanabe (1997) Bioorganic and Medicinal Chemistry 5: 437-444; now called rosuvastatin, see Olsson (2001) American Journal of Cardiology, 87, supplement 1, 33-36. The term “drug transportable by OATPC” means that transport by OATPC in humans is an important part of a drug exerting its pharmceutical effect in man. For example, some statins have to be transported to the liver by OATPC to exert their lipid lowering effects.

According to another aspect of the present invention there is provided a method of treating a human in need of treatment with a drug transportable by OATPC in which the method comprises:

diagnosis of a single nucleotide polymorphism in OATPC gene in the human, which diagnosis preferably comprises determining the sequence of the nucleic acid at position 487 in the coding sequence of the OATPC gene as defined by the position in SEQ ID NO: 1. and determining the status of the human by reference to polymorphism in the OATPC gene; and

administering an effective amount of the drug.

Although the polymorphism at position 487 (A→G; Asn130Asp) was reported by Tamai, no functional effect was attributed. Indeed the following polymorphisms are known but have not previously been attributed a role in statin pharmacogenetics as disclosed herein.

OATPC Polymorphisms

Position of nucleotide as in sequence 1 of OATPC patent

Position of amino acid as in sequence 2 of OATPC patent

| Exon | nucleotide | SNP | Amino acid | Change | Change |
|---|---|---|---|---|---|
| 4 | 487 | A > G | 130 | Asn > Asp | N > D |
| 4 | 554 | G > A | 152 | Arg > Lys | R > K |
| 5 | 620 | T > C | 174 | Val > Ala | V > A |
| 5 | 670 | C > T | 191 | Leu | L |
| 6 | 819 | A > T | 240 | Val | V |
| 6 | 820 | G > A | 241 | Asp > Asn | D > N |

According to another aspect of the present invention there is provided use of a drug transportable by OATPC in preparation of a medicament for treating a disease in a human diagnosed as having a polymorphism therein, preferably at one or more of the positions defined herein. Preferably the disease is cardiovascular.

According to another aspect of the present invention there is provided a pharmaceutical pack comprising OATPC transportable drug and instructions for administration of the drug to humans diagnostically tested for a single nucleotide polymorphism therein, preferably at one or more of the positions defined herein.

According to another aspect of the present invention there is provided an allelic variant of human OATPC polypeptide comprising at least one of the following:

a leucine at position 400 of SEQ ID NO 2;

an isoleucine at position 405 of SEQ ID NO 2;

an arginine at position 488 of SEQ ID NO 2;

a phenylalanine at position 643 of SEQ ID NO 2;

or a fragment thereof comprising at least 10 amino acids provided that the fragment comprises at least one allelic variant.

Fragments of polypeptide are at least 10 amino acids, more preferably at least 15 amino acids, more preferably at least 20 amino acids.

According to another aspect of the present invention there is provided an antibody specific for an allelic variant of human OATPC polypeptide as described herein.

Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the allelic variant of OATPC with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. No. RE 32,011, U.S. Pat. Nos. 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following Examples. All temperatures are in degrees Celsius.

In the Examples below, unless otherwise stated, the following methodology and materials have been applied.

AMPLITAQ™, available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989).

Electropherograms were obtained in a standard manner: data was collected by ABI377 data collection software and the wave form generated by ABI Prism sequencing analysis (2.1.2).

### EXAMPLE 1

Identification of Polymorphisms

1. Methods

DNA Preparation

DNA was prepared from frozen blood samples collected in EDTA following protocol I (Molecular Cloning: A Laboratory Manual, p392, Sambrook, Fritsch and Maniatis, 2^{nd} Edition, Cold Spring Harbor Press, 1989) with the following modifications. The thawed blood was diluted in an equal volume of standard saline citrate instead of phosphate buffered saline to remove lysed red blood cells. Samples were extracted with phenol, then phenol/chloroform and then chloroform rather than with three phenol extractions. The DNA was dissolved in deionised water.

Template Preparation

Templates were prepared by PCR using the oligonucleotide primers and annealing temperatures set out below. The extension temperature was 72° and denaturation temperature 94°. Generally 50 ng of genomic DNA was used in each reaction and subjected to 35 cycles of PCR. Where described below, the primary fragment was diluted 1/100 and two microlitres were used as template for amplification of secondary fragments. PCR was performed in two stages (primary fragment then secondary fragment) to ensure specific amplification of the desired target sequence.

Polymorphisms in OATPC: cDNA Screening of 15 Liver Samples

| | | | Amino Acid | Allele |
|---|---|---|---|---|
| Region | SNP | Position | Change | frequencies |
| Exon 4 | G/A | 510 | None | G = 96.7% |
| | | | | A = 3.3% |
| Exon 5 | C/T | 670 | None | C = 50% |
| | | | | T = 50% |
| Exon 5 | C/T | 696 | None | C = 60% |
| | | | | T = 40% |
| Exon 9 | C/G | 1299 | Phe 400 Leu | C = 96.7% |
| | | | | G = 3.3% |
| Exon 9 | G/A | 1312 | Val 405 Ile | G = 96.7% |
| | | | | A = 3.3% |
| Exon 9 | G/A | 1347 | None | G = 96.7% |
| | | | | A = 3.3% |
| Exon 10 | G/C | 1561 | Gly 488 Arg | G = 96.7% |
| | | | | C = 3.3% |
| Exon 14 | A/C | 2028 | Leu 643 Phe | A = 90% |
| | | | | C = 10% |

OATP2 above refers to the clone sequenced by Hsiang et al (ref 1). Some comment on the numbering of exons in OATPC is required. This gene contains an exon (38 bp) upstream (5′ UTR region) of the exon containing the ATG start site for translation. Therefore the exon numbering could vary depending whether this exon is counted as the first exon or not. In the literature, Konig (2000) JBC 275: 23161-68, have defined exon 1 as that containing the ATG start site and therefore we have adopted the same numbering in this application (but note that the priority document relating to the present application did vice versa; for example, exon 5 in this application is equivalent to exon 6 in the priority document).

PCR Products

| Fragment | Forward Oligo | Reverse Oligo |
|---|---|---|
| 443-999 | 443-466 | 979-999 |
| 874-1360 | 874-896 | 1337-1360 |
| 1255-1684 | 1255-1278 | 1663-1684 |
| 1559-2095 | 1559-1581 | 2073-2095 |

RFLP Analysis

| | | RFLP | |
|---|---|---|---|
| Polymorphism | Position | Enzyme/PCR size | RFLP fragment size |
| G/A | 510 | | |
| C/T | 670 | BmR I/595 bp | C = 349 bp, 246 bp |
| | | | T = 595 bp |
| C/T | 696 | | |
| C/G | 1299 | Apo I/595 bp | C = 30 bp, 60 bp, 380 bp |
| | | | G = 90 bp, 380 bp |
| G/A | 1312 | Bst 4CI/595 bp | G = 77 bp, 393 bp |
| | | | A = 470 bp |
| G/A | 1347 | | |
| G/C | 1561 | HpyCH4IV/595 bp | G = 470 bp |
| | | | C = 144 bp, 326 bp |
| A/C | 2028 | Ase I/577 bp | A = 89 bp, 488 bp |
| | | | C = 577 bp |

### EXAMPLE 2

Further OATPC Polymorphisms

SNPs in OATPC 3′UTR (positions according to SEQ ID NO 1)

| | | | Frequency | Frequency |
|---|---|---|---|---|
| Exon | Nucleotide | SNP | Caucasian | Japanese |
| 3′UTR | 2327 | Ins T | not screened | 0.1 |
| 3′UTR | 2342 | T > C | not screened | 0.4 |

SNPs in OATPC promoter (positions according to SEQ ID NO 3)

| | | Frequency | Frequency |
|---|---|---|---|
| Nucleotide | SNP | Caucasian | Japanese |
| 321 | T > G | 0.03 | not screened |
| 1332 | A > C | 0.08 | not screened |

SNPs in OATPC introns

| | Nucleotide | | Nucleotide | |
|---|---|---|---|---|
| | position in | | position | Sequence |
| Intron | relation to exon | SNP | in sequence | ID No |
| 1 | IVS1 + 21 | T > A | 41 | 4 |
| 2 | IVS2 + 89 | T > G | 109 | 5 |
| 2 | IVS2 + 224 | A > G | 244 | 5 |
| 3 | IVS3 + 97 | C > A | 117 | 6 |
| 3 | IVS3 + 263 | G > A | 283 | 6 |
| 4 | IVS4 + 189 | G > A | 209 | 7 |
| 4 | IVS4 + 191 | G > A | 211 | 7 |
| 4 | IVS5 − 118 | delCTTGTA | 63 | 8 |
| 6 | IVS6 + 33 | C > T | 53 | 9 |
| 9 | IVS10 − 107 | ins TTC | 75 | 10 |
| 11 | IVS11 + 142 | Ins T | 162 | 11 |
| 12 | IVS13 − 97 | G > C | 84 | 12 |

OATPC Intronic SNPs

Key

20 bp of exon sequence shown in uppercase Intron sequence in lowercase (200 to 300 bp only) SNP shown in uppercase (one allele only)

| Sequence ID No 4 | |
|---|---|
| IVS1+21 T>A SNP at position 41 in this sequence | |
| GATACTGCAA TGGATTGAAG gtagaataag ttttatgttt Ttgagctaaa ataagtaaat | 60 |
| agggaacttt aatgtataga aaagcaagtt gttaaaaaga acattatgtt tcaaattata | 120 |
| attttcaatt gaagcatata ttgaaatatt aacataatga ttcatacctt gatttaaacc | 180 |
| agtcttttaa tctgattaag | 200 |
| Sequence ID No 5 | |
| IVS2+89 T>G SNP at position 109 in this sequence | |
| IVS2+224 A>G SNP at position 244 in this sequence | |
| TGACGGAAGC TTTGAAATTG gtaacattta ttttctattt taataaccaa acttgcaaag | 60 |
| ttaaaaaata tatatgcttt acaccactgg ttatcaactg gggtaaatTt atctctcaca | 120 |
| ggcaatttgg caataactaa aaacatttgt ggttgtcata actgcacagg ggttgggggc | 180 |
| aatggaagtg ctactggtat ctaaaggtag aggtcagggg tactgctaaa tattctataa | 240 |
| tgcAcaaaga atgatgtaac tgaaaatgtt gatagtgagg atgttcagaa accctgattc | 300 |
| Sequence ID No 6 | |
| IVS3+97 C>A SNP at position 117 in this sequence | |
| IVS3+263 G>A SNP at position 283 in this sequence | |
| CCACATTTCT TCATGGGATA gtaagtgtta aaaaaaaaaa aaacctctgt gccactatca | 60 |
| gtaccttgta aattaggagt agaattttat tattatccct ttaaataggc agttacCttt | 120 |
| tgagaagata cccactaagt gtgtacagaa atgaaatagt gtctatttgt ctacataatc | 180 |
| attttattta tcgtagcttt catatacttt gaaataacaa aaagactaaa ctgtagagtt | 240 |
| tcaaatgaaa taaataggct ttttatgaat ttttagtata acGtatatac tgtacgtctt | 300 |
| Sequence ID No 7 | |
| IV54+189 G>A SNP at position 209 in this sequence | |
| IV54+191 G>A SNP at position 211 in this sequence | |
| ACCTGAGATA GTGGGAAAAG gtaagaatta atattgacag taaaaagtct tctaaaatgt | 60 |
| atacatttaa ttacatctct aaaaattgtt gtgatattca ttagcaaaat ttaattaaga | 120 |
| atgaatagga aaaacatttg actcttacag acataattat agtgttaata tacacagttc | 180 |
| gcccattaac aacacaggtt taaactacGc Gttttcactt ctatgcaaat tttgtccatc | 240 |
| tgaactggat gataaacctg ccggtaagaa tatctgacat tttctatatt tggattgaac | 300 |
| Sequence ID No 8 | |
| IVS5-118 delCTTGTA deletion of 6bp from position 63 to 68 incl. | |
| tagcagcata agaatggact aatacaccat attgtcaaag tttgcaaagt gaatataaat | 60 |
| taCTTGTAct tgtaaattaa aaaaaaataa gtagaataat taagagttta caagtagtta | 120 |
| aatttgtaat agaaatgcta aaattaatgt ttaaaatgaa acactctctt atctacatag | 180 |
| GTTGTTTAAA GGAATCTGGG | 200 |
| Sequence ID No 9IVS6+33 C>T SNP at position 53 in this | |
| sequence | |
| TATTGGATAT GTAGATCTAA gtaagtacaa ccagaacaag gtaccatgat aaCgtctttc | 60 |
| taagcacaca tgcgaaaaac attttttcaa ataactgaat tcactctttc aatagtcctt | 120 |
| tgcttaatat aattagaaag ttacaagtag gaaataaatg tattactaat cagaataaat | 180 |
| ataaaatcca gctcctattt | 200 |
| Sequence ID No 10 | |
| IVS10-107 Ins TTC SNP at position 75 in this sequence | |
| ttaaaaaaaa ctttgccatt tcgtcatcat caaagcaaat ttcttcatat aaagaaaaat | 60 |
| tctttatcta cttt(TTC)ttttcc ctctttctct gctttcactt tacttcttcc ttctcctccc | 120 |
| cttctttgtc tttttcttct ctctctctct ttttgatata tgtctatcat atatttccag | 180 |
| AAATAATCCA GTGACATCTC | 200 |
| Sequence ID No 11 | |
| IVS11+142 InsT InsT at position 162 in this sequence | |
| CATGTCATGC TGATTGTTAA gtaagtatga cttttaaaaa cattttcata tgcatgagac | 60 |
| tataaacaca cctaatgata tgcatatttt tacataatat actgggaatt caaattcata | 120 |
| tttcatcaaa ttttaatttt ctgagaattc attttattaa aa(T)ttactatg aactctcaag | 180 |
| gctgtaatta ataattttgc | 200 |
| Sequence ID No 12 | |
| 1VS13-97 G’C SNP at position 84 in this sequence | |
| tgatttgggt ctttgagatt tctaataatc tttattattg ggtagatgca gaacaaaata | 60 |
| ataaacgaat cctccaaatt tttGaacttt tatttaatca aaatatatca atgtggaata | 120 |
| tcatgcagtt acatttaaaa tatgttccct aaactgacat cttctcttct cctattacag | 180 |
| GAGGAATTCT AGCTCCAATA | 200 |

OATPC Promoter Region

Total length of the sequence=1538 bp

1500 bp of OATPC sequence directly upstream from the cDNA sequence

Sequence in uppercase represents 38 bp overlap with the cDNA sequence (SEQ ID NO 1) where this 38 bp is 5′ UTR sequence.

Nucleotide positions in the promoter have been determined where the −1 position is the base (lowercase) directly upstream of the end of the cDNA sequence.

| | |
|---|---|
| atgctctttg acctctgaaa atattggaga attttacaac tggcaccttt agctcaggat | 60 |
| tataaaggtt gttagttagt ttgtactgtt ttatcttcat tgtatataat atatatatta | 120 |
| gtctccaaac atgttgatgt gttttcaatg aaatggatgt ctgaggagaa aaccattagc | 180 |
| ctgagaaaac ccaaactqta ttcccattgt gaataaaagg aagtccataa aaatgatgga | 240 |
| aaatgttctg cattcctgtt atgatatcaa aatctggcag tacatgaaaa tttttcaaag | 300 |
| tgcttattta acaggcataa tctttggtct cctgagccag aatctgctgg gtatgggact | 360 |
| ggattgctat tttgacaact cgccagtaga ttcttactca gcagagtatt tggaagcctt | 420 |
| actctaatat tttggccttg ggtctacatt tctcagttct gcacagtcat tcttcccctc | 480 |
| tacactactc tttagtttgt ctcatgattc caatactctc aataattaac caagaataga | 540 |
| actaatcaat cagataactg tggcacagac atcaaataca ttttgctgca accatatcaa | 600 |
| caaatgtccc atgaatgata aggggtaacc atattctcat atatgcatcc tcacattacc | 660 |
| acatatatat atgtgcatat gtgtatacag gtaaaagtgt gtatatatgt atacatgtat | 720 |
| gtttgtgtgt atatacatac atatatcttc acacttttct gaaatatata tatttatgtg | 780 |
| agagaagggt ctgtacttta tttcagaaga gagcttaatg tccaaggtat aattgagagt | 840 |
| ctaaaatqtt tgagttattg aattaattaa acttcatctc tactcaagaa aacttttaac | 900 |
| tgagttaagc tcttcctttc tccacaagtc aagtcaataa aaggaaactg tgatattaat | 960 |
| aattctttcc tgttttgatg taaagaatct atcgcataaa gcagtcttaa ttttcatcat | 1020 |
| tcagaaaaat ggtcttgcag ttaattggga ctctcttatt ccaggtggta tctccagtct | 1080 |
| coatacatac cacgttagaa ccatacttat gtaccaagca aagagggtat attttaattt | 1140 |
| ttaaatgcca atgtaacctg taggcatatt ttttatttgt cttaaattat ttcctatttg | 1200 |
| gaagttttaa atacctggaa taatttattg tactcatatt tttaaagaaa aaaatcttat | 1260 |
| gccaccaact taattgaata aacaagtaaa agccattccc aaaagtaagg tttacttgtt | 1320 |
| aagattaaca aaaaataatg tgagaattct gagaaatata atctttaaat attggcaact | 1380 |
| ggagtgaact cttaaaacta actaggtttt atatgtttga ctagagcaat gacataataa | 1440 |
| ggtggttaat catcactgga cttgttttca aaaagccaac tactttaaga ggaataaagg | 1500 |
| GTGGACTTGT TGCAGTTGCT GTAGGATTCT AAATCCAG | 1538 |

1 A method for the detection of a polymorphism in OATPC in a human, which method comprises determining the sequence of the human at at least one of the following polymorphic positions:
positions 510, 696, 1299, 1312, 1347, 1561, 2028, 2327 and 2342 in sequence of the OATPC gene as defined by the position in SEQ ID NO: 1;
positions 400, 405, 488 and 643 in OATPC polypeptide defined by position in SEQ ID NO: 2;
positions 321 and 1332 defined by position in SEQ ID NO 3;
position 41 defined by position in SEQ ID NO 4;
positions 109 and 244 defined by position in SEQ ID NO 5;
positions 117 and 283 defined by position in SEQ ID NO 6;
positions 209 and 211 defined by position in SEQ ID NO 7;
positions 63 to 68 defined by position in SEQ ID NO 8;
position 53 defined by position in SEQ ID NO 9;
position 75 defined by position in SEQ ID NO 10;
position 162 defined by position in SEQ ID NO 11; and
positions 84 defined by position in SEQ ID NO 12.

2 Use of a method as defined in claim 1 to assess the pharmacogenetics of a drug transportable by OATPC.

3 A polynucleotide comprising at least 20 bases of the human OATPC gene and comprising an allelic variant selected from any one of the following:

| | | Position in | |
|---|---|---|---|
| Region | variant | SEQ ID NO | SEQ ID NO |
| Exon 4 | A | 510 | 1 |
| Exon 5 | T | 670 | 1 |
| Exon 5 | T | 696 | 1 |
| Exon 9 | G | 1299 | 1 |
| Exon 9 | A | 1312 | 1 |
| Exon 9 | A | 1347 | 1 |
| Exon 10 | C | 1561 | 1 |
| Exon 14 | C | 2028 | 1 |
| 3′ UTR | Insert T | 2327 | 1 |
| 3′ UTR | C | 2342 | 1 |
| Promoter | G | 321 | 3 |
| Promoter | C | 1332 | 3 |
| Intron 1 | A | 41 | 4 |
| Intron 2 | G | 109 | 5 |
| Intron 2 | G | 244 | 5 |
| Intron 3 | A | 117 | 6 |
| Intron 3 | A | 283 | 6 |
| Intron 4 | A | 209 | 7 |
| Intron 4 | A | 211 | 7 |
| Intron 4 | Deletion | 63 | 8 |
| | CTTGTA | | |
| Intron 6 | T | 53 | 9 |
| Intron 9 | Insert TTC | 75 | 10 |
| Intron 11 | Insert T | 162 | 11 |
| Intron 12 | C | 84 | 12 |

4 A nucleotide primer which can detect a polymorphism as defined in claim 1.

5 An allele specific primer capable of detecting a OATPC gene polymorphism as defined in claim 1.

6 An allele-specific oligonucleotide probe capable of detecting a OATPC gene polymorphism as defined in claim 1.

7 Use of an OATPC polymorphism as defined in claim 1 as a genetic marker in a linkage study.

8 A method of treating a human in need of treatment with a drug transportable by OATPC in which the method comprises:
i) detection of a polymorphism in OATPC in the human, which detection comprises determining the sequence of the human at one or more of the following positions:
positions 487, 510, 554, 670, 696, 819, 820, 1299, 1312, 1347, 1561, 2028, 2327 and 2342 in sequence of the OATPC gene as defined by the position in SEQ ID NO: 1;
positions 130, 152, 174, 241, 400, 405, 488 and 643 in OATPC polypeptide defined by position in SEQ ID NO: 2;
positions 321 and 1332 defined by position in SEQ ID NO 3;
position 41 defined by position in SEQ ID NO 4;
positions 109 and 244 defined by position in SEQ ID NO 5;
positions 117 and 283 defined by position in SEQ ID NO 6;
positions 209 and 211 defined by position in SEQ ID NO 7;
positions 63 to 68 defined by position in SEQ ID NO 8;
position 53 defined by position in SEQ ID NO 9;
position 75 defined by position in SEQ ID NO 10;
position 162 defined by position in SEQ ID NO 11; and
positions 84 defined by position in SEQ ID NO 12;
and determining the status of the human by reference to polymorphism in the OATPC gene; and
ii) administering an effective amount of the drug.

9 A method according to claim 8 wherein the drug is a statin.

10 A method according to claim 8 wherein the drug is rosuvastatin.

11 An allelic variant of human OATPC polypeptide comprising at least one of the following:
a leucine at position 400 of SEQ ID NO 2;
an isoleucine at position 405 of SEQ ID NO 2;
an arginine at position 488 of SEQ ID NO 2;
a phenylalanine at position 643 of SEQ ID NO 2;
or a fragment thereof comprising at least 10 amino acids provided that the fragment comprises at least one allelic variant.

12 An antibody specific for an allelic variant of human OATPC polypeptide as defined in claim 11.


SEQUENCE LISTING (12 sequences)

SEQ ID NO: 1 - DNA, 2452 bp, Homo sapiens
gtggacttgttgcagttgctgtaggattctaaatccaggtgattgtttcaaactgagcatcaacaacaaaaacatttgtatgatatctatatttcaatcatggaccaaaatcaacatttgaataaaacagcagaggcacaaccttcagagaataagaaaacaagatactgcaatggattgaagatgttcttggcagctctgtcactcagctttattgctaagacactaggtgcaattattatgaaaagttccatcattcatatagaacggagatttgagatatcctcttctcttgttggttttattgacggaagctttgaaattggaaatttgcttgtgattgtatttgtgagttactttggatccaaactacatagaccaaagttaattggaatcggttgtttcattatgggaattggaggtgttttgactgctttgccacatttcttcatgggatattacaggtattctaaagaaactaatatcaattcatcagaaaattcaacatcgaccttatccacttgtttaattaatcaaattttatcactcaatagagcatcacctgagatagtgggaaaaggttgtttaaaggaatctgggtcatacatgtggatatatgtgttcatgggtaatatgcttcgtggaataggggagactcccatagtaccactggggctttcttacattgatgatttcgctaaagaaggacattcttctttgtatttaggtatattgaatgcaatagcaatgattggtccaatcattggctttaccctgggatctctgttttctaaaatgtacgtggatattggatatgtagatctaagcactatcaggataactcctactgattctcgatgggttggagcttggtggcttaatttccttgtgtctggactattctccattatttcttccataccattctttttcttgccccaaactccaaataaaccacaaaaagaaagaaaagcttcactgtctttgcatgtgctggaaacaaatgatgaaaaggatcaaacagctaatttgaccaatcaaggaaaaaatattaccaaaaatgtgactggttttttccagtcttttaaaagcatccttactaatcccctgtatgttatgtttgtgcttttgacgttgttacaagtaagcagctatattggtgcttttacttatgtcttcaaatacgtagagcaacagtatggtcagccttcatctaaggctaacatcttattgggagtcataaccatacctatttttgcaagtggaatgtttttaggaggatatatcattaaaaaattcaaactgaacaccgttggaattgccaaattctcatgttttactgctgtgatgtcattgtccttttacctattatattttttcatactctgtgaaaacaaatcagttgccggactaaccatgacctatgatggaaataatccagtgacatctcatagagatgtaccactttcttattgcaactcagactgcaattgtgatgaaagtcaatgggaaccagtctgtggaaacaatggaataacttacatctcaccctgtctagcaggttgcaaatcttcaagtggcaataaaaagcctatagtgttttacaactgcagttgtttggaagtaactggtctccagaacagaaattactcagcccatttgggtgaatgcccaagagatgatgcttgtacaaggaaattttacttttttgttgcaatacaagtcttgaatttatttttctctgcacttggaggcacctcacatgtcatgctgattgttaaaattgttcaacctgaattgaaatcacttgcactgggtttccactcaatggttatacgagcactaggaggaattctagctccaatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaaggggagaaaaaaagccacttctgcttctgtgtttccaaacagcattgcattgattcagtaagatgttatttttgaggagttcctggtcctttcactaagaatttccacatcttttatggtggaagtataaataagcctatgaacttataataaaacaaactgtaggtagaaaaaatgagagtactcattgtacattatagctacatatttgtggttaaggttagactatatgatccatacaaattaaagtgagagacatggttactgtgtaataaaa

SEQ ID NO: 2 - protein, 691 aa, Homo sapiens
MDQNQHLNKTAEAQPSENKKTRYCNGLKMFLAALSLSFIAKTLGAIIMKSSIIHIERRFEISSSLVGFIDGSFEIGNLLVIVFVSYFGSKLHRPKLIGIGCFIMGIGGVLTALPHFFMGYYRYSKETNINSSENSTSTLSTCLINQILSLNRASPEIVGKGCLKESGSYMWIYVFMGNMLRGIGETPIVPLGLSYIDDFAKEGHSSLYLGILNAIAMIGPIIGFTLGSLFSKMYVDIGYVDLSTIRITPTDSRWVGAWWLNFLVSGLFSIISSIPFFFLPQTPNKPQKERKASLSLHVLETNDEKDQTANLTNQGKNITKNVTGFFQSFKSILTNPLYVMFVLLTLLQVSSYIGAFTYVFKYVEQQYGQPSSKANILLGVITIPIFASGMFLGGYIIKKFKLNTVGIAKFSCFTAVMSLSFYLLYFFILCENKSVAGLTMTYDGNNPVTSHRDVPLSYCNSDCNCDESQWEPVCGNNGITYISPCLAGCKSSSGNKKPIVFYNCSCLEVTGLQNRNYSAHLGECPRDDACTRKFYFFVAIQVLNLFFSALGGTSHVMLIVKIVQPELKSLALGFHSMVIRALGGILAPIYFGALIDTTCIKWSTNNCGTRGSCRTYNSTSFSRVYLGLSSMLRVSSLVLYIILIYAMKKKYQEKDINASENGSVMDEANLESLNKNKHFVPSAGADSETHC

SEQ ID NO: 3 - DNA, 1538 bp, Homo sapiens
atgctctttgacctctgaaaatattggagaattttacaactggcacctttagctcaggattataaaggttgttagttagtttgtactgttttatcttcattgtatataatatatatattagtctccaaacatgttgatgtgttttcaatgaaatggatgtctgaggagaaaaccattagcctgagaaaacccaaactgtattcccattgtgaataaaaggaagtccataaaaatgatggaaaatgttctgcattcctgttatgatatcaaaatctggcagtacatgaaaatttttcaaagtgcttatttaacaggcataatctttggtctcctgagccagaatctgctgggtatgggactggattgctattttgacaactcgccagtagattcttactcagcagagtatttggaagccttactctaatattttggccttgggtctacatttctcagttctgcacagtcattcttcccctctacactactctttagtttgtctcatgattccaatactctcaataattaaccaagaatagaactaatcaatcagataactgtggcacagacatcaaatacattttgctgcaaccatatcaacaaatgtcccatgaatgataaggggtaaccatattctcatatatgcatcctcacattaccacatatatatatgtgcatatgtgtatacaggtaaaagtgtgtatatatgtatacatgtatgtttgtgtgtatatacatacatatatcttcacacttttctgaaatatatatatttatgtgagagaagggtctgtactttatttcagaagagagcttaatgtccaaggtataattgagagtctaaaatgtttgagttattgaattaattaaacttcatctctactcaagaaaacttttaactgagttaagctcttcctttctccacaagtcaagtcaataaaaggaaactgtgatattaataattctttcctgttttgatgtaaagaatctatcgcataaagcagtcttaattttcatcattcagaaaaatggtcttgcagttaattgggactctcttattccaggtggtatctccagtctccatacataccacgttagaaccatacttatgtaccaagcaaagagggtatattttaatttttaaatgccaatgtaacctgtaggcatattttttatttgtcttaaattatttcctatttggaagttttaaatacctggaataatttattgtactcatatttttaaagaaaaaaatcttatgccaccaacttaattgaataaacaagtaaaagccattcccaaaagtaaggtttacttgttaagattaacaaaaaataatgtgagaattctgagaaatataatctttaaatattggcaactggagtgaactcttaaaactaactaggttttatatgtttgactagagcaatgacataataaggtggttaatcatcactggacttgttttcaaaaagccaactactttaagaggaataaagggtggacttgttgcagttgctgtaggattctaaatccag

SEQ ID NO: 4 - DNA, 200 bp, Homo sapiens
gatactgcaatggattgaaggtagaataagttttatgtttttgagctaaaataagtaaatagggaactttaatgtatagaaaagcaagttgttaaaaagaacattatgtttcaaattataattttcaattgaagcatatattgaaatattaacataatgattcataccttgatttaaaccagtcttttaatctgattaag

SEQ ID NO: 5 - DNA, 300 bp, Homo sapiens
tgacggaagctttgaaattggtaacatttattttctattttaataaccaaacttgcaaagttaaaaaatatatatgctttacaccactggttatcaactggggtaaatttatctctcacaggcaatttggcaataactaaaaacatttgtggttgtcataactgcacaggggttgggggcaatggaagtgctactggtatctaaaggtagaggtcaggggtactgctaaatattctataatgcacaaagaatgatgtaactgaaaatgttgatagtgaggatgttcagaaaccctgattc

SEQ ID NO: 6 - DNA, 300 bp, Homo sapiens
ccacatttcttcatgggatagtaagtgttaaaaaaaaaaaaaacctctgtgccactatcagtaccttgtaaattaggagtagaattttattattatccctttaaataggcagttaccttttgagaagatacccactaagtgtgtacagaaatgaaatagtgtctatttgtctacataatcattttatttatcgtagctttcatatactttgaaataacaaaaagactaaactgtagagtttcaaatgaaataaataggctttttatgaatttttagtataacgtatatactgtacgtctt

SEQ ID NO: 7 - DNA, 300 bp, Homo sapiens
acctgagatagtgggaaaaggtaagaattaatattgacagtaaaaagtcttctaaaatgtatacatttaattacatctctaaaaattgttgtgatattcattagcaaaatttaattaagaatgaataggaaaaacatttgactcttacagacataattatagtgttaatatacacagttcgcccattaacaacacaggtttaaactacgcgttttcacttctatgcaaattttgtccatctgaactggatgataaacctgccggtaagaatatctgacattttctatatttggattgaac

SEQ ID NO: 8 - DNA, 200 bp, Homo sapiens
tagcagcataagaatggactaatacaccatattgtcaaagtttgcaaagtgaatataaattacttgtacttgtaaattaaaaaaaaataagtagaataattaagagtttacaagtagttaaatttgtaatagaaatgctaaaattaatgtttaaaatgaaacactctcttatctacataggttgtttaaaggaatctggg

SEQ ID NO: 9 - DNA, 200 bp, Homo sapiens
tattggatatgtagatctaagtaagtacaaccagaacaaggtaccatgataacgtctttctaagcacacatgcgaaaaacattttttcaaataactgaattcactctttcaatagtcctttgcttaatataattagaaagttacaagtaggaaataaatgtattactaatcagaataaatataaaatccagctcctattt

SEQ ID NO: 10 - DNA, 203 bp, Homo sapiens
ttaaaaaaaactttgccatttcgtcatcatcaaagcaaatttcttcatataaagaaaaattctttatctactttttcttttccctctttctctgctttcactttacttcttccttctcctccccttctttgtctttttcttctctctctctctttttgatatatgtctatcatatatttccagaaataatccagtgacatctc

SEQ ID NO: 11 - DNA, 201 bp, Homo sapiens
catgtcatgctgattgttaagtaagtatgacttttaaaaacattttcatatgcatgagactataaacacacctaatgatatgcatatttttacataatatactgggaattcaaattcatatttcatcaaattttaattttctgagaattcattttattaaaatttactatgaactctcaaggctgtaattaataattttgc

SEQ ID NO: 12 - DNA, 200 bp, Homo sapiens
tgatttgggtctttgagatttctaataatctttattattgggtagatgcagaacaaaataataaacgaatcctccaaatttttgaacttttatttaatcaaaatatatcaatgtggaatatcatgcagttacatttaaaatatgttccctaaactgacatcttctcttctcctattacaggaggaattctagctccaata